(19) United States
(12) Patent Application Publication
(10) Pub. No.: US 2023/0390774 A1
(43) Pub. Date: Dec. 7, 2023

(54) CONTAINER FOR A SAMPLE AND METHOD FOR EXAMINING A SAMPLE WITH THE CONTAINER

(71) Applicant: Paul Marienfeld GmbH & Co. KG, Lauda-Konigshofen (DE)

(72) Inventor: Gerd MARIENFELD, Lauda-Konigshofen (DE)

(21) Appl. No.: 18/452,841

(22) Filed: Aug. 21, 2023

Related U.S. Application Data
(63) Continuation of application No. PCT/EP2022/054511, filed on Feb. 23, 2022.

(30) Foreign Application Priority Data
Feb. 24, 2021 (EP) 21158992.4

(51) Int. Cl. B01L 3/00; G01N 21/03

(52) U.S. Cl. CPC B01L 3/508; G01N 21/03; G01N 2021/0378; B01L 2300/0858

(57) ABSTRACT

The invention relates to a container for a sample. In order to improve an optical or spectroscopic examination of a sample stored in the container, a container is proposed, comprising a bottom portion, a container axis being oriented perpendicularly to the bottom portion; a first wall portion, said first wall portion adjoining the bottom portion; and a second wall portion, said second wall portion adjoining said first wall portion, and wherein the second wall portion is inclined by at least 20° with respect to the container axis.

### CROSS-REFERENCE TO RELATED APPLICATIONS

This is a bypass continuation application of International PCT Application No. PCT/EP2022/054511, filed on Feb. 23, 2022, which claims priority to European Patent Application No. 21158992.4, filed on Feb. 24, 2021, which are incorporated by reference herein in their entirety.

### BACKGROUND

The disclosure relates to a container for a sample, in particular for a biological sample. Furthermore, the disclosure relates to a method for examining a sample and a use of a container for storing a sample during an examination.

To examine tissue samples, tissue may be removed from the body of living beings (e.g. humans or animals) and the tissue may be processed and then optically examined, for example. Processing may include dehydrating and clearing the tissue, wherein alcohol, which is often used to dehydrate tissue, may be removed during the clearing process. The processed sample may be divided into slices with a thickness of a few microns, which are then placed or mounted on a slide. This sample may also be colored. The slide with the sample may then be placed in a container and covered with a liquid. The sample may also be glued directly to the bottom of the container. The container may be covered with a (thin) glass pane. The sectioned and colored tissue sample may then be examined optically, for example by microscopy with transmitted light or fluorescence. The examination takes place outside the body of the living being. However, no information about the three-dimensional position of sections of the sample can be obtained by this examination, i.e., it is not possible to determine a three-dimensional position of the cells within the tissue or components of the tissue.

When a liquid is stored in a container, there is an interaction between the liquid and the surface of the container wall. Depending on the liquid and the material of the container wall, a curvature of the liquid surface (concave or convex) forms in the contact area between the liquid and the container wall. The curved surface of the liquid—also called a concave meniscus—acts as a lens by which an examination of a sample in the container covered by the liquid may be disturbed or falsified.

### SUMMARY

The invention is therefore based on the object of providing a container which improves an optical or spectroscopic examination of a sample stored in the container, in particular when the sample is covered with a liquid and when the radiation source and the radiation receiver are not in a line with the sample.

Another object of the invention is to provide a container which allows for an examination of a plurality of planes of a sample in the container, thereby allowing for a three-dimensional examination to be carried out.

Yet another object is to provide a container that can be manufactured cost-effectively.

The object is achieved by the subject matter and methods of the independent claims. Advantageous developments are specified in the dependent claims.

A container for a sample comprises a bottom portion, a first wall portion and a second wall portion. A container axis is oriented or located perpendicular to the bottom portion. The first wall portion adjoins the bottom portion. The second wall portion adjoins the first wall portion. The second wall portion is inclined by at least 20° with respect to the container axis. That is, the container has a flat bottom surface which is adjoined by a first wall portion which encloses the bottom surface and projects upwards. The first wall portion thus forms a first receiving space with the bottom surface. A second wall portion, which has a different inclination than the first wall portion, adjoins this first wall portion. The second wall portion thus encloses a second receiving space above the first receiving space.

A container for a sample may also comprise a bottom portion, a first wall portion and a second wall portion. A container axis is oriented or located perpendicular to the bottom portion. The first wall portion adjoins the base portion and the second wall portion adjoins the first wall portion. A cross-sectional area of the container at half of the height of the first wall portion may be smaller (by at least 5% or at least 10%) than the cross-sectional area of the container at half of the height of the second wall portion. Half of the height may be half the total height of the respective wall portion in the direction of the container axis.

That is, the container has a flat bottom surface, which is adjoined by a first wall portion which encloses the bottom surface and projects upwards. The first wall portion thus forms a first receiving space with the bottom surface. A second wall portion, which has a different inclination than the first wall portion, adjoins this first wall portion. The second wall portion thus encloses a second receiving space above the first receiving space.

Due to the configuration of the second wall portion, a liquid can be introduced into the container in such a way that an incline of the liquid surface that is disruptive to an optical or spectroscopic examination is outside the area under observation. As described above, the sample may be adhered to a slide which is then placed in the container.

In a plane which is parallel to the bottom portion or perpendicular to the container axis and which is delimited by the first wall portion (at least on one side), the liquid surface may be substantially (±5°) flat or substantially (±5°) uncambered or substantially (±5°) parallel to the bottom portion or substantially (±5°) perpendicular to the container axis. This allows for the sample to be positioned on the entire bottom portion without interfering with the examination. The sample may thus rest in the first receiving space and be surrounded by liquid. The liquid is filled up to the second receiving space. Possible menisci therefore form only in the edge area of the second receiving space and cannot have a disruptive effect when the sample is irradiated for optical examination.

The sample may be a biological sample, for example a tissue sample. The sample may be a mixture of substances, for example a homogeneous or heterogeneous mixture of substances. The sample may be a pure substance.

The bottom portion may have an area of at least 200 mm², in particular the bottom portion has an area of at least 400 mm² or at least 500 mm². The area of the bottom portion may be at most 5000 mm², preferably at most 4000 mm², more preferably at most 3000 mm², more preferably at most 2500 mm². The area of the bottom portion is particularly preferably between 200 mm 2 and 5000 mm².

At least the surface of the bottom portion facing toward the interior of the container may be configured to be planar over its entire area.

The first wall portion may be formed integrally with the bottom portion.

An angle may be formed between the first wall portion and the container axis. The first wall portion may be inclined by this angle with respect to the container axis. The angle between the container axis and the first wall portion may be at least 0.5°. The angle between the container axis and the first wall portion is preferably at least 1.0° or at least 1.5°. The angle between the container axis and the first wall portion may be at most 50°, preferably at most 40°, more preferably at most 30°, more preferably at most 25°, more preferably at most 10°, more preferably at most 5°. In particular, the angle between the container axis and the first wall portion is between 0.5° and 50°, preferably between 0.5° and 30°, more preferably between 0.5° and 10°. The angle between the container axis and the first wall portion is particularly preferably between 1.0° and 3.0° or between 1.5° and 2.5°.

No angle may be formed between the first wall portion and the container axis. The first wall portion may be oriented in parallel to the container axis. An angle between the first wall portion and the bottom portion may be substantially 90°.

The first wall portion may have a height (in the direction of the container axis) of at least 5 mm, preferably at least 7 mm, more preferably at least 10 mm, more preferably at least 15 mm, more preferably at least 24 mm. The height of the first wall portion may be at most 100 mm, preferably at most 80 mm, more preferably at most 60 mm, more preferably at most 50 mm, more preferably at most 30 mm, more preferably at most 25 mm or 15 mm. The height of the first wall portion may be between 5 mm and 100 mm, preferably between 7 mm and 60 mm.

The first wall portion may have a width (perpendicular to the direction of the container axis) of at least 5 mm, more preferably at least 10 mm, preferably at least 20 mm, more preferably at least 30 mm, more preferably at least 35 mm or 40 mm. The width of the first wall portion may be at most 150 mm, preferably at most 130 mm, more preferably at most 110 mm, more preferably at most 90 mm, more preferably at most 70 mm, more preferably at most 50 mm. The width of the first wall portion may be between 5 mm and 150 mm, preferably between 10 mm and 100 mm, more preferably between 30 mm and 60 mm. The width may be a maximum width of the first wall portion.

The first wall portion can have a width that is greater than its height by at least 5 mm.

The first wall portion may be configured to be planar.

The second wall portion may be formed integrally with the first wall portion.

An angle is formed between the second wall portion and the container axis. The second wall portion is inclined by this angle with respect to the container axis. The angle may be at least 30°, preferably at least 40°, more preferably at least 50°, more preferably at least 55°, more preferably at least 60°. The angle between the second wall portion and the container axis may be at most 90°. The angle between the second wall portion and the container axis is preferably at most 80°, more preferably at most 75°. The angle between the second wall portion and the container axis may be between 20° and 90°, preferably between 30° and 80°, more preferably between 40° and 70°.

The second wall portion may have a height (in the direction of the container axis) of at least 0.5 mm, preferably at least 1 mm, more preferably at least 1.5 mm. The height of the second wall portion may be at most 30 mm, preferably at most 20 mm, more preferably at most 15 mm, more preferably at most 10 mm, more preferably at most 5 mm. The height of the second wall portion may be between 0.5 mm and 30 mm, preferably between 1 mm and 20 mm.

The second wall portion may have a width (perpendicular to the direction of the container axis) of at least 5 mm, more preferably at least 10 mm, preferably at least 20 mm, more preferably at least 30 mm, more preferably at least 40 mm or 50 mm. The width of the second wall portion may be at most 150 mm, preferably at most 130 mm, more preferably at most 110 mm, more preferably at most 90 mm, more preferably at most 70 mm, more preferably at most 60 mm. The width of the second wall portion may be between 5 mm and 150 mm, preferably between 10 mm and 100 mm, more preferably between 40 mm and 70 mm. The width may be a maximum width of the first wall portion.

A third wall portion may adjoin the second wall portion. The third wall portion may be formed integrally with the second wall portion. The third wall portion thus encloses a third receiving space arranged above the second receiving space.

An angle may be formed between the third wall portion and the container axis. The third wall portion may be inclined by said angle with respect to the container axis. The angle may be at least 1°, preferably at least 2°, more preferably at least 3°, more preferably at least 4°, more preferably at least 5°. The angle between the third wall portion and the container axis may be at most 50°. The angle between the third wall portion and the container axis is preferably at most 40°, more preferably at most 30°, more preferably at most 20°, more preferably at most 10°, more preferably at most 8°. The angle between the third wall portion and the container axis may be between 1° and 50°, preferably between 2° and 30°, more preferably between 3° and 10°.

The third wall portion may have a height (in the direction of the container axis) of at least 1 mm, preferably at least 5 mm, more preferably at least 8 mm. The height of the third wall portion may be at most 100 mm, preferably at most 50 mm, more preferably at most 30 mm, more preferably at most 20 mm, more preferably at most 15 mm. The height of the third wall portion may be between 1 mm and 100 mm, preferably between 1 mm and 50 mm, more preferably between 5 mm and 20 mm.

The third wall portion may have a width (perpendicular to the direction of the container axis) of at least 5 mm, more preferably at least 10 mm, more preferably at least 20 mm, more preferably at least 30 mm, more preferably at least 40 mm or 50 mm. The width of the third wall portion may be at most 150 mm, preferably at most 130 mm, more preferably at most 110 mm, more preferably at most 90 mm, more preferably at most 80 mm, more preferably at most 70 mm. The width of the third wall portion may be between 5 mm and 150 mm, preferably between 10 mm and 100 mm, more preferably between 40 mm and 70 mm. The width may be a maximum width of the first wall portion.

The container may have an opening opposed to the bottom portion. The opening may be defined by the third wall portion or by third wall portions. The opening may have a larger area than the bottom portion. The large-area opening (relative to the bottom portion) of the container may ensure that an imaging at the bottom of the container can utilize the numerical aperture of an objective above a sample in the container as optimally as possible. Due to the configuration of the container, the path of light emitted by the sample is impeded as little as possible. If appropriate, the lens may be lowered into the container, in particular into the third receiving space, which is formed by the third wall portion.

The (angles or inclinations of the) wall portions of the wall side or the wall sides may be selected in such a way that a numerical aperture of the lens objective can be utilized, in particular can be utilized as optimally as possible.

The edge of the opening of the container may have a constant height (in the direction of the axis of the container). A plane defined by the opening of the container may be parallel to the bottom portion.

The second wall portion may adjoin the first wall portion in the direction of the container axis. The third wall portion may adjoin to the second wall portion in the direction of the container axis.

The first wall portion may have a different inclination with respect to the container axis than the second wall portion. The second wall portion may have a different inclination with respect to the container axis than the third wall portion.

The described first wall portion, second wall portion and/or third wall portion may be wall portions of a first wall side of the container. The first wall side may consist of the first, second and third wall portions or include said wall portions.

The container, in particular the bottom portion, may be a polygonal basic shape having the first wall side, a second wall side, a third wall side and a fourth wall side.

The second wall side may include or consist of a first wall portion, a second wall portion and a third wall portion. The first wall portion, the second wall portion and the third wall portion of the second wall side may be equal to the first wall portion, the second wall portion and the third wall portion of the first wall side, respectively.

The third wall side may include or consist of a first wall portion, a second wall portion and a third wall portion. The first wall portion, the second wall portion and the third wall portion of the third wall side may be equal to the first wall portion, the second wall portion and the third wall portion of the first wall side, respectively.

The fourth wall side may include or consist of a first wall portion, a second wall portion and a third wall portion. The first wall portion, the second wall portion and the third wall portion of the fourth wall side can be equal to the first wall portion, the second wall portion and the third wall portion of the first wall side, respectively.

The polygonal basic shape may be a rectangular basic shape, in particular a square basic shape. The basic polygonal shape is preferably rectangular, but not square. Likewise, the basic polygonal shape may be an n-gon, where n is an integer and is greater than four.

The basic shape of the container, in particular the bottom portion, may also correspond to an ellipse, in particular a circle, with a chord. The chord may correspond to the first side of the wall. The light entry point is then in the area of the chord, i.e. in the planar area.

Similarly, the first wall portion, the second wall portion and the third wall portion of the third wall side may have a different inclination (with respect to the container axis) than the first wall portion, the second wall portion and the third wall portion of the first wall side, respectively.

The first wall portion of the third wall side may have a greater inclination with respect to the container axis than the first wall portion of the first wall side. The first wall portion of the third wall side is preferably at least 2° or at least 5° more inclined with respect to the container axis than the first wall portion of the first wall side.

The first wall portion of the third wall side may be at most 30°, preferably at most 20°, more preferably at most 15°, more inclined with respect to the container axis than the first wall portion of the first wall side.

The second wall portion of the third wall side may have a smaller inclination with respect to the container axis than the second wall portion of the first wall side. The second wall portion of the third wall side is preferably at least 2° or at least 5° less inclined with respect to the container axis than the second wall portion of the first wall side.

The second wall portion of the third wall side may be at most 30°, preferably at most 20°, more preferably at most 15° less inclined than the second wall portion of the first wall side with respect to the container axis.

The first wall portion, the second wall portion and the third wall portion of the second wall side may have a different inclination (relative to the container axis) than the first wall portion, the second wall portion and the third wall portion of the first wall side, respectively.

The first wall portion of the second wall side may have a greater inclination with respect to the container axis than the first wall portion of the first wall side. The inclination of the first wall portion of the second wall side with respect to the container axis is preferably at least 1° or at least 2° greater than the inclination of the first wall portion of the first wall side with respect to the container axis.

The first wall portion of the second wall side may be at most 20°, preferably at most 10°, more preferably at most 5° more inclined with respect to the container axis than the first wall portion of the first wall side.

Similarly, the second wall portion of the second wall side may have a smaller inclination with respect to the container axis than the second wall portion of the first wall side. The inclination of the second wall portion of the second wall side with respect to the container axis is preferably at least 1° or at least 2° less than the inclination of the second wall portion of the first wall side with respect to the container axis.

The second wall portion of the second wall side may be at most 20°, preferably at most 10°, more preferably at most 5° less inclined than the first wall portion of the first wall side with respect to the container axis.

The first wall portion, the second wall portion and the third wall portion of the fourth wall side may have a different inclination (with respect to the container axis) than the first wall portion, the second wall portion and the third wall portion of the first wall side, respectively.

The first wall portion of the fourth wall side may have a greater inclination with respect to the container axis than the first wall portion of the first wall side. The inclination of the first wall portion of the fourth wall side with respect to the container axis is preferably at least 1° or at least 2° greater than the inclination of the first wall portion of the first wall side with respect to the container axis.

The first wall portion of the fourth wall side may be at most 20°, preferably at most 10°, more preferably at most 5°, more inclined with respect to the container axis than the first wall portion of the first wall side.

The second wall portion of the fourth wall side may have a smaller inclination with respect to the container axis than the second wall portion of the first wall side. The inclination of the second wall portion of the fourth wall side with respect to the container axis is preferably at least 1° or at least 2° less than the inclination of the second wall portion of the first wall side with respect to the container axis.

The second wall portion of the fourth wall side may be at most 20°, preferably at most 10°, more preferably at most 5° less inclined than the first wall portion of the first wall side with respect to the container axis.

A container for a sample may comprise a bottom portion and a first wall portion. A container axis is oriented or located perpendicular to the bottom portion. The first wall portion adjoins the bottom portion. The first wall portion is inclined by 1.9°±0.7° or n times that, where n is an integer and greater than 1, with respect to the container axis.

The inclination of the container axis with respect to the first wall portion of 1.9°±0.7° or n times that, where n is an integer and greater than 1, may also be applied to all containers described herein.

The inclination of the first wall portion of 1.9°±0.7° defines a range from 1.2° to 2.6°. n times that, n being an integer with n>1, corresponds to a multiplication of a value in the range by n. For example, 1.5° is in the range 1.9°±0.7° and n may be equal to 3, giving an inclination of 4.5°, or 2.2° is in the range 1.9°±0.7° and n may be equal to 3, giving a tilt of 6.6°.

The first wall portion is preferably inclined with respect to the container axis by 1.9°±0.5° or n times that, where n is an integer and greater than 1. More preferably, the first wall portion is inclined by 1.9°±0.3° or n times that, where n is an integer greater than 1, with respect to the container axis. More preferably, the first wall portion is inclined with respect to the container axis by 1.9°±0.15° or n times that, where n is an integer greater than 1. The integer value for n may be at most ten, preferably at most seven, more preferably at most five, more preferably at most three.

The container may be an injection molded container. The container may have been produced by injection molding.

The entire container may have been formed in one piece.

Preferably, the material of the container is transparent. The container may be completely transparent. All wall sides of the container may be transparent. At least one wall side of the container may be transparent, preferably at least two wall sides of the container are transparent. At least the first wall portion of the container may be transparent. In particular, at least the first wall portion of the first wall side is transparent. Alternatively or additionally, the first wall portion of the second wall side may be transparent. Alternatively or additionally, the first wall portion of the third wall side may be transparent. Alternatively or additionally, the first wall portion of the fourth wall side may be transparent. The container may be transparent at least at the point where the light is radiated into it.

The container may comprise a thermoplastic or consist of a thermoplastic. The container may comprise or consist of one or more materials. The first wall portion of the first wall side may comprise a different material or consist of a different material than the second and/or third wall portion of the first wall side. Alternatively or additionally, the first wall portion of the second wall side may comprise a different material or consist of a different material than the second and/or third wall portion of the second wall side. Alternatively or additionally, the first wall portion of the third wall side may comprise a different material or consist of a different material than the second and/or third wall portion of the third wall side. Alternatively or additionally, the first wall portion of the fourth wall side may comprise a different material or consist of a different material than the second and/or third wall portion of the fourth wall side.

The container may comprise one or more pigments. In particular, the container comprises a pigment suitable for laser marking. A pigment suitable for laser marking may change color when irradiated with a laser.

The container may be made of a material suitable for manufacture by injection molding.

An opening of the container may be defined by the third wall portion or portions.

The container may have a wall thickness between 0.5 mm and 5.0 mm, preferably between 1.0 mm and 4.0 mm, more preferably between 1.0 mm and 2.0 mm.

The container may have a height of less than 150 mm, preferably less than 120 mm, more preferably less than 100 mm, more preferably less than 80 mm, more preferably less than 50 mm, more preferably less than 30 mm, more preferably less than 25 mm, more preferably less than 15 mm. In particular, the height of the container is at least 5 mm or at least 10 mm.

The container may hold a volume of less than 100 ml, preferably less than 75 ml, more preferably less than 50 ml, more preferably less than 30 ml. Preferably, the container may hold a volume of at least 5 ml, in particular at least 10 ml. In particular, the container may hold a volume between 5 ml and 50 ml.

The third wall side may be opposite the first wall side. The fourth wall side may be opposite the second wall side.

The container may comprise a polymer (thermoplastic) or consist of the polymer. The polymer may be a copolymer, in particular a block copolymer.

The polymer may contain units obtained by (complete) hydrogenation of polystyrene units. The polymer may comprise polycyclohexylethene units.

The polymer may contain units obtained by (complete) hydrogenation of polybutadiene units. The polymer may comprise ethylene-1-butene copolymer units.

The polymer may have been obtained or prepared by (complete) hydrogenation of a styrene-butadiene copolymer.

The polymer may be a block copolymer, in particular a cyclic block copolymer.

The polymer may be transparent, in particular in a wavelength range from 380 nm to 780 nm.

A portion of the container can consist of a material, preferably the (entire) container consists of the material.

The material may have a density of at most 1.00 g cm⁻³, preferably at most 0.98 g cm⁻³, more preferably at most 0.96 g cm⁻³, most preferably between 0.92 g cm⁻³ and 0.98 g cm⁻³. The density may be determined according to ASTM D792.

The material may have a water absorption of less than 5%, preferably less than 2.0%, more preferably less than 0.5%. Water absorption may be determined according to ASTM D570.

The material may have a melt flow rate of at least 1.0 cm⁻³ 10 min⁻¹, preferably at least 7.0 cm⁻³ 10 min⁻¹, more preferably at least 100.0 cm⁻³ 10 min⁻¹, more preferably between 2.0 cm⁻³ 10 min⁻¹ and 250.0 cm⁻³ 10 min⁻¹. In particular, the melt flow rate of the material is between 5.0 cm⁻³ 10 min⁻¹ and 20.0 cm⁻³ 10 min⁻¹. The melt flow rate may be determined according to ASTM D1238 with a weight of 2.6 kg and a temperature of 260° C.

The material may have a transmittance of at least 50%, preferably at least 70%, more preferably at least 85%, more preferably at least 90%. The transmittance at 3 mm may be determined according to ASTM D1003.

The material may have a transmittance at a wavelength of 488 nm and at a wavelength of 638 nm of at least 50%, preferably at least 60%, more preferably at least 70%, more preferably between 80% and 85%.

The material may have a haze of less than 5.0%, preferably less than 3.0%, more preferably less than 1.5%, more preferably less than 1.0. Haze at 3 mm may be determined according to ASTM D1003.

The material may have a refractive index of less than 5.0, preferably less than 3.0, more preferably less than 2.5, more preferably less than 2.0, more preferably less than 1.6, more preferably between 1.46 and 1.56.

The material may have a Vicat softening point of at least 70° C., preferably at least 85° C., more preferably at least 100° C., more preferably between 90° C. and 130° C. In particular, the Vicat softening point is between 118° C. and 128° C. The Vicat softening point may be determined according to ASTM D1525 at 1 kg and 50° C. h⁻¹.

The material may have a heat deflection temperature of at least 50° C., preferably at least 60° C., more preferably at least 75° C., more preferably between 60° C. and 115° C. In particular, the heat deflection temperature is between 97° C. and 108° C. The heat deflection temperature may be determined according to ASTM D648 at 0.455 MPa and 2° C. min⁻¹.

The material may have a flexural strength of at least 30 MPa, preferably at least 40 MPa, more preferably at least 50 MPa, more preferably between 40 MPa and 80 MPa. In particular, the flexural strength is between 65 MPa and 75 MPa. The flexural strength may be determined according to ASTM D790.

The material may have a flexural modulus of at least 600 MPa, preferably at least 1000 MPa, more preferably at least 1400 MPa, more preferably between 1000 MPa and 3000 MPa. In particular, the flexural modulus is between 2200 MPa and 2600 MPa. The flexural modulus may be determined according to ASTM D790.

The material may have a tensile strength (Y.P. or B.P.) of at least 15 MPa, preferably at least 25 MPa, more preferably at least 30 MPa, more preferably between 20 MPa and 50 MPa. In particular, the tensile strength (Y.P. or B.P.) is between 35 MPa and 48 MPa. Tensile strength (Y.P. or B.P.) may be determined according to ASTM D638.

The material may have a strain of at least 5%, preferably at least 8%, more preferably at least 10%, more preferably between 8% and 40%. In particular, the strain of the material is between 20% and 29%. Strain may be determined according to ASTM D638.

The material may have an Izod impact strength of at least 1 kg-cm cm⁻¹, preferably at least 1.5 kg-cm cm⁻¹, more preferably at least 2.0 kg-cm cm⁻¹, more preferably between 1.5 kg-cm cm⁻¹ and 5.0 kg-cm cm⁻¹. In particular, the Izod impact strength is between 2.8 kg-cm cm⁻¹ and 4.4 kg-cm cm⁻¹. The Izod impact strength may be determined according to ASTM D256.

The material may be chemically resistant to methyl salicylate (2-hydroxybenzoic acid methyl ester), dibenzyl ether (1,1′-[oxybis(methylene)]bisbenzene), a mixture of one part benzyl alcohol (phenylmethanol) and two parts benzyl benzoate (benzoic acid phenylmethyl ester), a mixture of four parts benzyl benzoate (phenoxybenzene, 1,1′-oxybisbenzene) and one part diphenyl ether, ethyl cinnamate (ethyl 3-phenylprop-2-enoate), and/or 2,2′ thiodiethanol (thiodiglycol, bis(2-hydroxyethyl)sulfide). Chemical resistance may be determined by submerging a specimen for examining tensile strength (e.g., vie ASTM D638) of the material or granules of the material in the liquid (solvent) for two days (48 h or seven days) at room temperature (23° C.) (so that it is covered by the liquid). After two days, the weight loss is determined and, if the weight loss is less than 5.0%, preferably less than 1.0%, the material is chemically resistant to the liquid. The strain at break may also be determined after these two days (e.g. according to ASTM D638) and, if the elongation at break decreases by less than 50%, preferably less than 10%, compared to a test specimen not submerged in the liquid, the material is chemically resistant to the liquid.

The material may have an autofluorescence, based on the autofluorescence of polystyrene, of at most 50%, preferably at most 40%, more preferably at most 30%, more preferably at most 25%. The autofluorescence at an excitation wavelength of 350 nm (sample thickness 1 mm) may be determined for the material and for polystyrene and the ratio of the values for the determined autofluorescence may be taken (autofluorescence material/autofluorescence polystyrene). Autofluorescence may be considered in a wavelength range from 400 nm to 460 nm. In particular, the autofluorescence at a wavelength of 420 nm may be considered, wherein the autofluorescence of the material, based on the autofluorescence of polystyrene, is at most 50%, preferably at most 40%, more preferably at most 30%, more preferably at most 20%, more preferably at most 15%.

When excited in a wavelength range from 400 nm to 900 nm, the material may show no (measurable) autofluorescence.

Each of the containers disclosed herein may be used in a method of examining a sample. The sample may be a biological sample. The sample is placed in the container. The sample is illuminated and an emission from the sample is recorded.

The sample may be illuminated by a radiation source, in particular by a laser.

The emission of the sample may be a fluorescence.

The sample may be illuminated through the first wall portion of the first wall side of the container.

The emission of the sample may be recorded substantially (±10°) perpendicular to the direction of illumination.

The emission of the sample may be collected through an objective.

Each of the containers disclosed herein may be stored for storage of a sample during examination of the sample.

The examination may be a microscopic examination, more preferably a light sheet microscopic examination, more preferably a light sheet fluorescence microscopic examination.

### BRIEF DESCRIPTION OF THE DRAWINGS

Exemplary embodiments of the disclosure are shown in the figures and are described in more detail below.

FIG. 1 shows an isometric view of a container 100.

FIG. 2 shows the container 100 in a view from below.

FIG. 3 shows a sectional view A-A of the container 100 as indicated in FIG. 2

FIG. 4 shows a sectional view B-B of the container 100 as indicated in FIG. 2

FIG. 5 shows an isometric view of a container 200.

FIG. 6 shows the container 200 in a view from below.

FIG. 7 shows a sectional view C-C of the container 200 as indicated in FIG. 6

FIG. 8 shows a sectional view D-D of the container 200 as indicated in FIG. 6

FIG. 9 shows a system 400 for measuring a sample.

### DETAILED DESCRIPTION

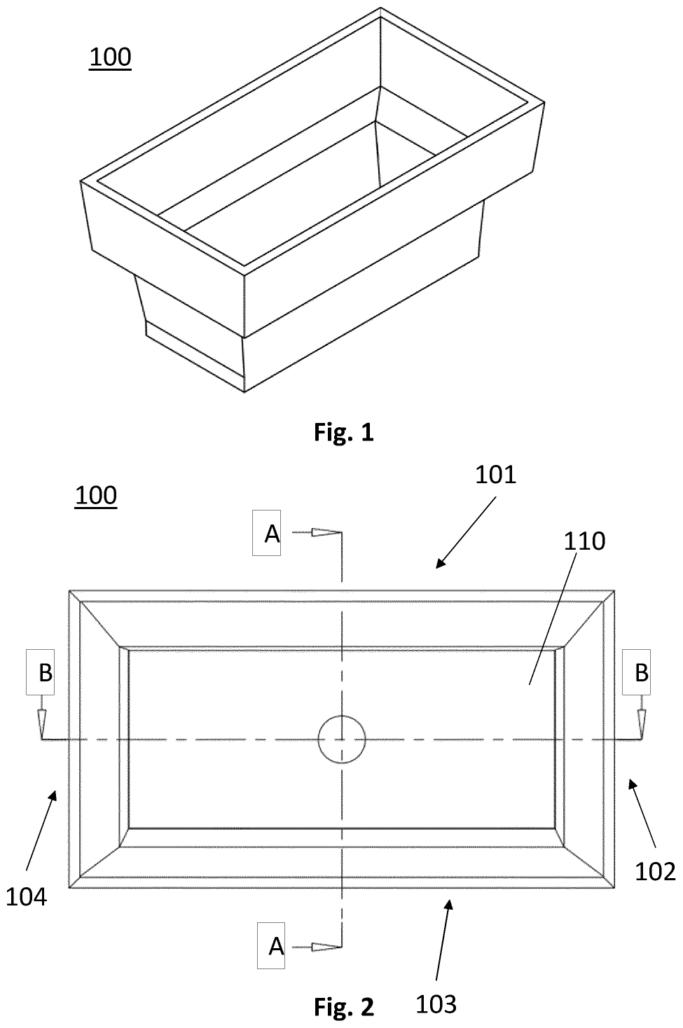

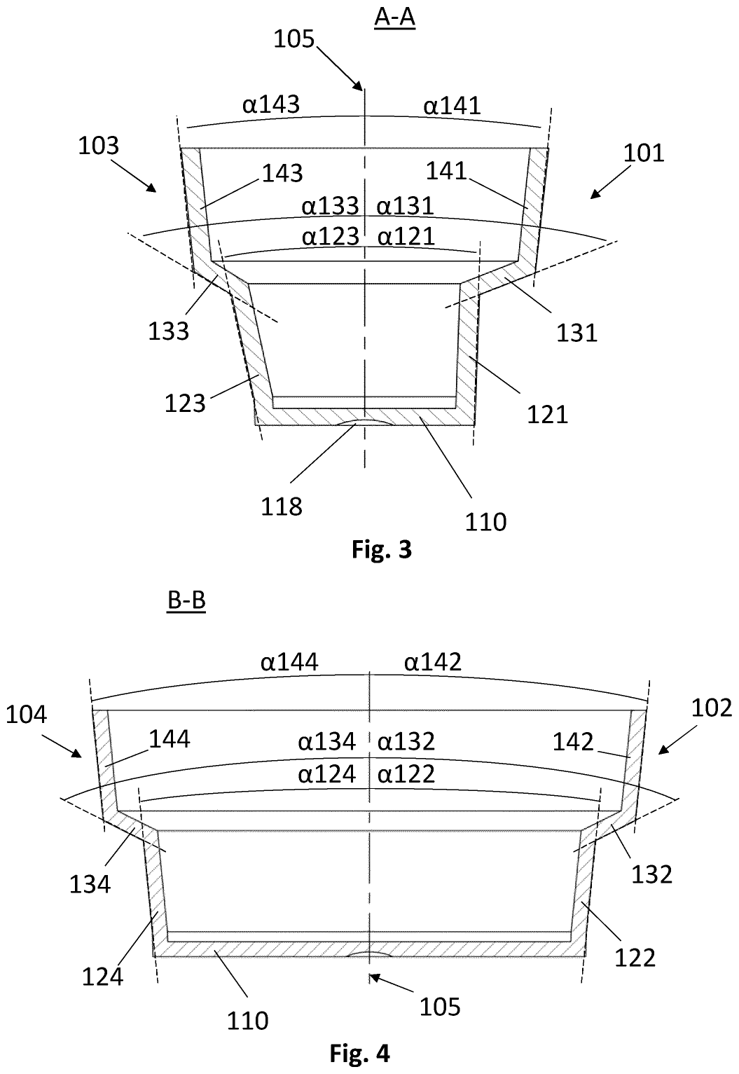

FIGS. 1 and 2 show a container 100. In FIG. 1, the container 100 is shown in an isometric view and in FIG. 2 the container 100 is shown in a view from below. In FIG. 2, the profile of two sections A-A and B-B is indicated. Sectional views A-A and B-B are shown in FIGS. 3 and 4.

The container 100 comprises a bottom portion 110 and a first wall side 101. The container 100 may comprise a second wall side 102, a third wall side 103 and a fourth wall side 104.

In general, the container 100 may have a polygonal basic shape. In particular, the bottom portion 110 of the container 100 may have a polygonal shape. The polygonal basic shape or polygonal shape is in particular rectangular.

The first wall side 101 and the third wall side 103 may have a greater width than the second wall side 102 and the fourth wall side 104. The width of the first wall side 101 and the third wall side 103 is preferably greater by a factor of at least 1.5 than the width of the second wall side 102 and the fourth wall side 104.

In the section A-A of FIG. 3, the first wall side 101, the bottom portion 110 and the third wall side 103 are shown in section.

The inner surface (facing the interior of the container) of the bottom portion 110 may be planar. A sprue cap 118 may be provided in the bottom portion 110 such that despite the sprue cap 118, the inner surface of the bottom portion 110 is planar. The sprue cap 118 may be provided when the container 100 is injection molded.

The bottom portion 110, e.g., the inner surface of the bottom portion 110, may define a container axis 105. The container axis may extend perpendicularly to the bottom portion 110. The bottom portion 110 may define a plane to which the container axis 105 is oriented perpendicularly.

The first wall side 101 may comprise a first wall portion 121, a second wall portion 131 and a third wall portion 141.

The first wall portion 121 may (directly) adjoin the bottom portion 110, in particular in the direction of the container axis 105. The second wall portion 131 may (directly) adjoin the first wall portion, in particular in the direction of the container axis 105. The third wall portion 141 may (directly) adjoin the second wall portion 131, in particular in the direction of the container axis 105.

An angle α121 may be formed between the container axis 105 and the first wall portion 121. The first wall portion 121 may be inclined by this angle α121 with respect to the container axis 105. The first wall portion 121 is preferably inclined outwards with respect to the container axis 105 (pointing away from the interior of the container 100).

The angle α121 may be 1.9°±0.7° or n times that, where n is an integer greater than 1. Preferably, the angle α121 is 1.9°±0.5° or n times that, where n is an integer and greater than 1, more preferably 1.9°±0.3°, or n times that, where n is an integer and is greater than 1, more preferably 1.90°±0.15°, or n times that, where n is an integer and greater than 1.

An angle α131 may be formed between the container axis 105 and the second wall portion 131. The second wall portion 131 may be inclined (outwards) by the angle α131 with respect to the container axis 105.

An angle α141 may be formed between the container axis 105 and the third wall portion 141. The third wall portion 141 may be inclined (outwards) by the angle α141 with respect to the container axis 105.

The first wall portion 121, the second wall portion 131 and the third wall portion 141 may each be inclined (outwards) with respect to the container axis 105.

The cross section (parallel to the bottom portion 110) of the container 100 may be smaller at the bottom portion 110 than the cross section of the container 100 in the first wall portion 121 (at medium height of the first wall portion 121 in the direction of the container axis 105). The cross section of the container 100 may be smaller in the first wall portion 121 (at medium height of the first wall portion 121 in the direction of the container axis 105) than the cross section of the container 100 in the second wall portion 131 (at medium height of the second wall portion 131 in the direction of the container axis 105). The cross section of the container 100 in the second wall portion 131 (at the medium height of the second wall portion 131 in the direction of the container axis 105) may be smaller than the cross section of the container 100 in the third wall portion 141 (at medium height of the third wall portion 141 in the direction of the container axis 105).

The cross section of the container 100 may increase in the direction of the container axis 105 (continuously, preferably with a non-constant slope). Due to the design of the container 100, a volume of liquid (volume of clearing agent) to be introduced for a sample examination may be small, so that liquid or clearing agent is saved.

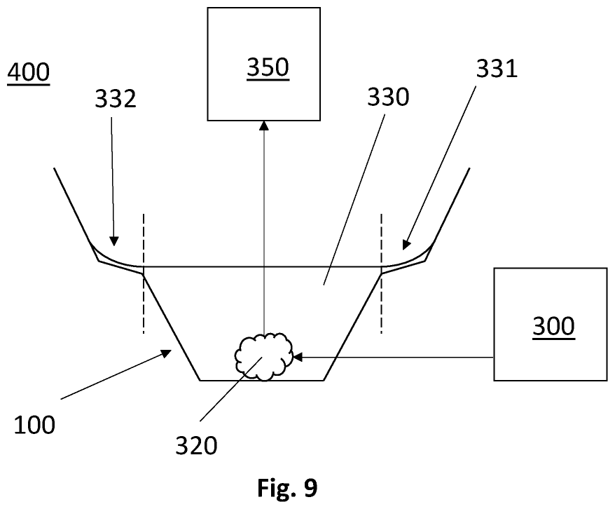

A user can grip the container 100 in the upper area (third wall portion or third wall portions). This reduces or avoids fingerprints or other surface contamination on the surface of the first wall portion or the first wall portions. As described with reference to FIG. 9, the first wall portion may be transilluminated by a radiation source.

Similarly, the upper area (third wall portion or third wall portions) of the container 100 may be provided with a label. The label may include a data matrix code and/or consecutive numbering. The label may include information about the container 100 and/or about the sample to be stored or stored therein and/or a liquid to be introduced or introduced into the container 100.

Between the first wall portion 121 and the second wall portion 131, there may be a kink in the inclination of the wall portions 121, 131 with respect to the container axis 105. The kink may result from a sudden or discontinuous change in the inclination with respect to the container axis 105. Between the second wall portion 131 and the third wall portion 141, there may be a kink in the inclination of the wall portions 131, 141 with respect to the container axis.

The kink between the first wall portion 121 and the second wall portion 131 and/or the kink between the second wall portion 131 and the third wall portion 141 may act as a marker for an intended filling level or quantity, for example of a clearing agent, of the container 100.

The angle α121 between the container axis 105 and the first wall portion 121 may be less than the angle α131 between the container axis 105 and the second wall portion 131. The angle α131 between the container axis 105 and the second wall portion 131 may be greater than the angle α141 between the container axis 105 and the third wall portion 141. The angle α121 between the container axis 105 and the first wall portion 121 may be less than the angle α141 between the container axis 105 and the third wall portion 141.

The third wall side 103 may comprise a first wall portion 123, a second wall portion 133 and a fourth wall portion 143.

The first wall portion 123 of the third wall side 103 may (directly) adjoin the bottom portion 110. The second wall portion 133 of the third wall side 103 may (directly) adjoin the first wall portion 133 of the third wall side 103. The third wall portion 143 of the third wall side 103 may (directly) adjoin the second wall portion 133 of the third wall side 103.

An angle α123 may be formed between the container axis 105 and the first wall portion 123 of the third wall side 103. The first wall portion 123 may be inclined by this angle α123 with respect to the container axis 105. The first wall portion 123 of the third wall side 103 is preferably inclined outwards relative to the container axis 105 (pointing away from the interior of the container 100).

An angle α133 may be formed between the container axis 105 and the second wall portion 133 of the third wall side 103. The second wall portion 133 of the third wall side 103 may be inclined (outwards) by the angle α133 with respect to the container axis 105.

An angle α143 may be formed between the container axis 105 and the third wall portion 143. The third wall portion 143 of the third wall side 103 may be inclined (outwards) by the angle α143 with respect to the container axis 105.

The first wall portion 123 of the third wall side 103, the second wall portion 131 of the third wall side 103 and the third wall portion 141 of the third wall side 103 may each be inclined (outward) with respect to the container axis 105.

Between the first wall portion 123 of the third wall side 103 and the second wall portion 133 of the third wall side 103, there may be a kink in the inclination of the wall portions 123, 133 with respect to the container axis 105. Between the second wall portion 133 of the third wall side 103 and the third wall portion 143 of the third wall side 103, there may be a kink in the inclination of the wall portions 133, 143 with respect to the container axis 105.

The angle α123 between the container axis 105 and the first wall portion 123 of the third wall side 103 may be less than the angle α133 between the container axis 105 and the second wall portion 133 of the third wall side 103. The angle α133 between the container axis 105 and the second wall portion 133 of the third wall side 103 may be greater than the angle α143 between the container axis 105 and the third wall portion 143 of the third wall side 103. The angle α123 between the container axis 105 and the first wall portion 123 may be greater than the angle α143 between the container axis 105 and the third wall portion 143 of the third wall side 103.

The first wall portion 121 of the first wall side 101 may be opposite the first wall portion 123 of the third wall side 103. The first wall portion 121 of the first wall side 101 may have substantially (±10%) the same height (in the direction of the container axis 105) as the first wall portion 123 of the third wall side 103.

The second wall portion 131 of the first wall side 101 may be opposite the second wall portion 133 of the third wall side 103. The second wall portion 131 of the first wall side 101 may have substantially (±10%) the same height as the second wall portion 133 of the third wall side 103.

The third wall portion 141 of the first wall side 101 may be opposite the third wall portion 143 of the third wall side 103. The third wall portion 141 of the first wall side 101 may substantially (±10%) the same height as the third wall portion 143 of the third wall side 103.

The angle α121 between the container axis 105 and the first wall portion 121 of the first wall side 101 may be less than the angle α123 between the container axis 105 and the first wall portion 123 of the third wall side 103.

The angle α131 between the container axis 105 and the second wall portion 131 of the first wall side 101 may be greater than the angle α123 between the container axis 105 and the second wall portion 133 of the third wall side 103.

FIG. 4 shows the section B-B, as indicated in FIG. 2. The bottom portion 110, the second wall side 102 and the fourth wall side 104 are sectioned.

The second wall side 102 may comprise a first wall portion 122, a second wall portion 132 and a third wall portion 142.

The first wall portion 122 of the second wall side 102 may (directly) adjoin the bottom portion 110, in particular in the direction of the container axis 105. The second wall portion 132 of the second wall side 102 may (directly) adjoin the first wall portion 122 of the second wall side 102, in particular in the direction of the container axis 105. The third wall portion 142 of the second wall side 102 may (directly) adjoin the second wall portion 132 of the second wall side 102, in particular in the direction of the container axis 105.

An angle α122 may be formed between the container axis 105 and the first wall portion 122 of the second wall side 102. The first wall portion 122 of the second wall side 102 may be inclined by this angle α122 with respect to the container axis 105. The first wall portion 122 of the second wall side 102 is preferably inclined outwards with respect to the container axis 105 (pointing away from the interior of the container 100).

An angle α132 may be formed between the container axis 105 and the second wall portion 132 of the second wall side 102. The second wall portion 132 of the second wall side 102 may be inclined (outwards) by the angle α132 with respect to the container axis 105.

An angle α142 may be formed between the container axis 105 and the third wall portion 142 of the second wall side 102. The third wall portion 142 of the second wall side 102 may be inclined (outwards) by the angle α142 with respect to the container axis 105.

The first wall portion 122 of the second wall side 102, the second wall portion 132 of the second wall side 102 and the third wall portion 142 of the second wall side 102 may each be inclined (outward) with respect to the container axis 105.

Between the first wall portion 122 of the second wall side 102 and the second wall portion 132 of the second wall side 102, there may be a kink in the inclination of the wall portions 122, 132 with respect to the container axis 105. Between the second wall portion 132 of the second wall side 102 and the third wall portion 142 of the second wall side 102, there may be a kink in the inclination of the wall portions 132, 142 with respect to the container axis 105.

The angle α122 between the container axis 105 and the first wall portion 122 of the second wall side 102 may be less than the angle α132 between the container axis 105 and the second wall portion 132 of the second wall side 102. The angle α132 between the container axis 105 and the second wall portion 132 of the second wall side 102 may be greater than the angle α142 between the container axis 105 and the third wall portion 142 of the second wall side 102. The angle α122 between the container axis 105 and the first wall portion 122 of the second wall side 102 may be less than the angle α142 between the container axis 105 and the third wall portion 142 of the second wall side 102.

The fourth wall side 104 may comprise a first wall portion 124, a second wall portion 134, and a third wall portion 144.

The first wall portion 124 of the fourth wall side 104 may (directly) adjoin the bottom portion 110, in particular in the direction of the container axis 105. The second wall portion 134 of the fourth wall side 104 may (directly) adjoin the first wall portion 124 of the fourth wall side 104, in particular in the direction of the container axis 105. The third wall portion 144 of the fourth wall side 104 may (directly) adjoin the second wall portion 134 of the fourth wall side 104, in particular in the direction of the container axis 105.

An angle α124 may be formed between the container axis 105 and the first wall portion 124 of the fourth wall side 104. The first wall portion 124 of the fourth wall side 104 may be inclined by this angle α124 with respect to the container axis 105. The first wall portion 124 of the fourth wall side 104 is preferably inclined outwards with respect to the container axis 105 (pointing away from the interior of the container 100).

An angle α134 may be formed between the container axis 105 and the second wall portion 134 of the fourth wall side 104. The second wall portion 134 of the fourth wall side 104 may be inclined (outwards) by the angle α134 with respect to the container axis 105.

An angle α144 may be formed between the container axis 105 and the third wall portion 144 of the fourth wall side 104. The third wall portion 144 of the fourth wall side 104 may be inclined (outwards) by the angle α144 with respect to the container axis 105.

The first wall portion 124 of the fourth wall side 104, the second wall portion 134 of the fourth wall side 104 and the third wall portion 144 of the fourth wall side 104 may each be inclined (outwards) with respect to the container axis 105.

Between the first wall portion 124 of the fourth wall side 104 and the second wall portion 134 of the fourth wall side 104, there may be a kink in the inclination of the wall portions 124, 134 with respect to the container axis 105. Between the second wall portion 134 of the fourth wall side 104 and the third wall portion 144 of the fourth wall side 104, there may<be a kink in the inclination of the wall portions 134, 144 with respect to the container axis 105.

The angle α124 between the container axis 105 and the first wall portion 124 of the fourth wall side 104 may be less than the angle α134 between the container axis 105 and the second wall portion 134 of the fourth wall side 104. The angle α134 between the container axis 105 and the second wall portion 134 of the fourth wall side 104 may be greater than the angle α144 between the container axis 105 and the third wall portion 144 of the fourth wall side 104. The angle α124 between the container axis 105 and the first wall portion 124 of the fourth wall side 104 may be less than the angle α144 between the container axis 105 and the third wall portion 144 of the fourth wall side 104.

The angle α122 between the container axis 105 and the first wall portion 122 of the second wall side 102 may have substantially (±2° or ±1°) the same value as the angle α124 between the container axis 105 and the first wall portion 124 of the fourth wall side 104. The angle α132 between the container axis 105 and the second wall portion 132 of the second wall side 102 may have substantially (±2° or ±1°) the same value as the angle α134 between the container axis 105 and the second wall portion 134 of the fourth wall side 104. The angle α142 between the container axis 105 and the third wall portion 142 of the second wall side 102 may have substantially (±2° or ±1°) the same value as the angle α144 between the container axis 105 and the third wall portion 144 of the fourth wall side 104.

The angle α122 between the container axis 105 and the first wall portion 122 of the second wall side 102 may be greater than the angle α121 between the container axis 105 and the first wall portion 121 of the first wall side 101.

The angle α124 between the container axis 105 and the first wall portion 124 of the fourth wall side 104 may be greater than the angle α121 between the container axis 105 and the first wall portion 121 of the first wall side 101.

The angle α132 between the container axis 105 and the second wall portion 132 of the second wall side 102 may be less than the angle α131 between the container axis 105 and the second wall portion 131 of the first wall side 101.

The angle α134 between the container axis 105 and the second wall portion 134 of the fourth wall side 104 may be less than the angle α131 between the container axis 105 and the second wall portion 131 of the first wall side 101.

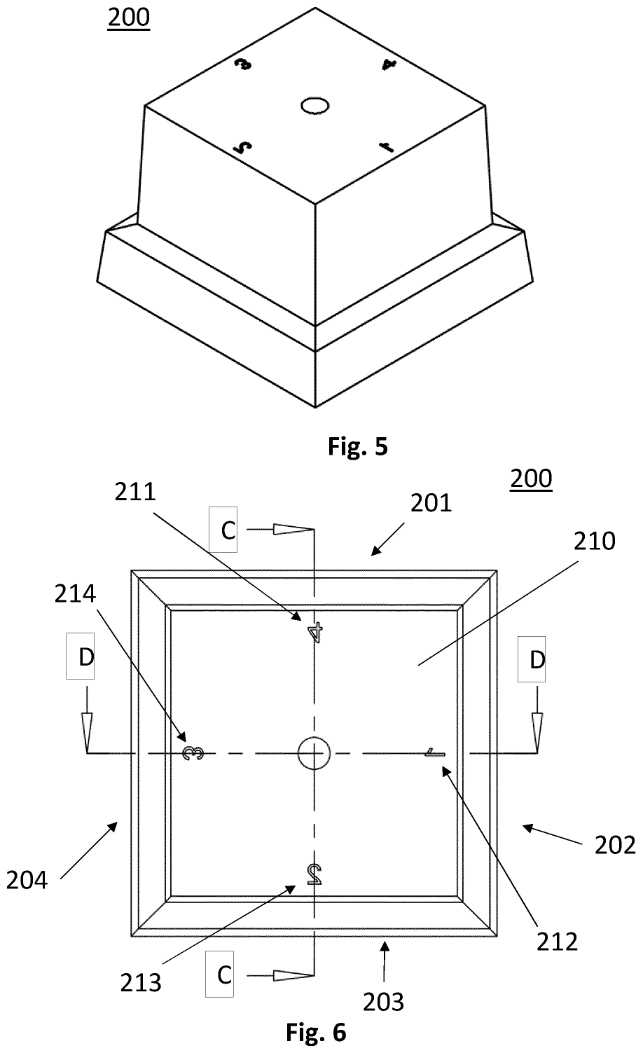

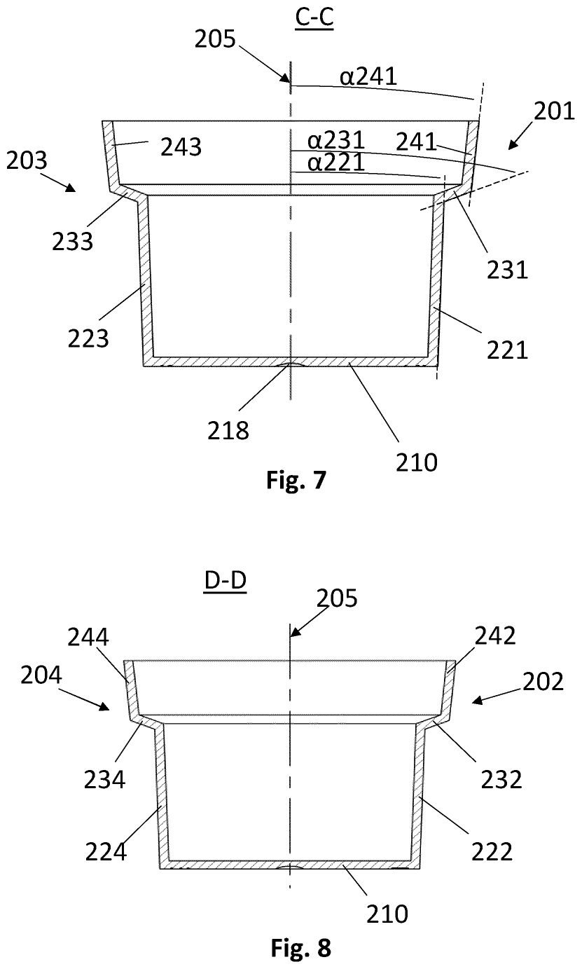

In FIGS. 5 and 6, a container 200 is shown. The container 200 is shown in an isometric view in FIG. 5 and the container 200 is shown in a bottom view in FIG. 6. Sections CC and D-D, which are shown in FIGS. 7 and 8, are indicated in FIG. 6.

The container 200 may comprise a bottom portion 210, a first wall side 201, a second wall side 202, a third wall side 203 and a fourth wall side 204.

The container 200 may generally have a polygonal basic shape, preferably the bottom portion 210 of the container 200 has a polygonal shape. The polygonal basic shape or polygonal shape may be square.

The container 200, particularly the bottom portion 210 of the container 200, may include markings 211,212,213,214. Each of the markings 211, 212, 213, 214 may be assigned to one of the wall sides 201, 202, 203, 204. The markings 211, 212, 213, 214 may be used to identify the wall sides 201, 202, 203, 204. For example, the markings 211, 212, 213, 214 may be consecutive numbers, e.g. 1, 2, 3, 4, or the markers 211, 212, 213, 214 may be consecutive letters, e.g. A, B, C, D.

FIG. 7 shows the section CC, wherein the bottom portion 210, the first wall side 201 and the third wall side 203 are sectioned.

The inner surface (facing the interior of the container 200) of the bottom portion 210 may be planar. A sprue cap 218 may be provided in the base portion 210 such that, despite the sprue cap 218, the inner surface of the base portion 210 is planar. The sprue cap 218 may be provided when the container 100 is injection molded.

The bottom portion 210, e.g. the inner surface of the bottom portion 210, may define a container axis 205. The container axis 205 may extend perpendicularly to the bottom portion 210. The bottom portion 210 may define a plane perpendicular to the container axis 205.

The first wall side 201 may comprise a first wall portion 221, a second wall portion 231, and a third wall portion 241.

The first wall portion 221 may (directly) adjoin the bottom portion 210, in particular in the direction of the container axis 205. The second wall portion 231 may (directly) adjoin the first wall portion 221, in particular in the direction of the container axis 205. The third wall portion 241 may (directly) adjoin the second wall portion 231, in particular in the direction of the container axis 205.

An angle α221 may be formed between the container axis 205 and the first wall portion 221. The first wall portion 221 may be inclined by this angle α221 with respect to the container axis 205. The first wall portion 221 is preferably inclined outwards with respect to the container axis 205 (pointing away from the interior of the container 200).

An angle α231 may be formed between the container axis 205 and the second wall portion 231. The second wall portion 231 may be inclined (outwards) by the angle α231 with respect to the container axis 205.

An angle α241 may be formed between the container axis 205 and the third wall portion 241. The third wall portion 241 may be inclined (outwards) by the angle α241 with respect to the container axis 205.

The first wall portion 221, the second wall portion 231, and the third wall portion 241 may each be inclined (outward) with respect to the container axis 205.

The cross section (parallel to the bottom portion 210) of the container 200 at the bottom portion 210 may be less than the cross section of the container 200 in the first wall portion 221 (at medium height of the first wall portion 221 in the direction of the container axis 205). The cross section of the container 200 in the first wall portion 221 (at medium height of the first wall portion 221 in the direction of the container axis 205) may be less than the cross section of the container 200 in the second wall portion 231 (at medium height of the second wall portion 231 in the direction of the container axis 205). The cross section of the container 200 in the second wall portion 231 (at medium height of the second wall portion 231 in the direction of the container axis 205) may be less than the cross section of the container 200 in the third wall portion 241 (at medium height of the third wall portion 241 in the direction of the container axis 205).

Between the first wall portion 221 and the second wall portion 231, there may be a kink in the inclination of the wall portions 221, 231 with respect to the container axis 205. The kink may result from a sudden or discontinuous change in the inclination with respect to the container axis 205. A kink in the inclination of the wall portions 231, 241 with respect to the container axis 205 may be present between the second wall portion 231 and the third wall portion 241.

The angle α221 between the container axis 205 and the first wall portion 221 may be less than the angle α231 between the container axis 205 and the second wall portion 231. The angle α231 between the container axis 205 and the second wall portion 231 may be greater than the angle α241 between the container axis 205 and the third wall portion 241. The angle α221 between the container axis 205 and the first wall portion 221 may be less than the angle α241 between the container axis 205 and the third wall portion 241.

The third wall side 203 may comprise a first wall portion 223, a second wall portion 233 and a fourth wall portion 243.

The first wall portion 223 of the third wall side 203 may (directly) adjoin the bottom portion 210. The second wall portion 233 of the third wall side 203 may (directly) adjoin the first wall portion 233 of the third wall side 203. The third wall portion 243 of the third wall side 203 may (directly) adjoin the second wall portion 233 of the third wall side 203.

The first wall portion 221 of the first wall side 201 is opposite the first wall portion 223 of the third wall side 203. The first wall portion 221 of the first wall side 201 may have substantially (±10%) the same height (in the direction of the container axis 205) as the first wall portion 223 of the third wall side 203.

The second wall portion 231 of the first wall side 201 may be opposite the second wall portion 233 of the third wall side 203. The second wall portion 231 of the first wall side 201 may have substantially (±10%) the same height as the second wall portion 233 of the third wall side 203.

The third wall portion 241 of the first wall side 201 may be opposite the third wall portion 243 of the third wall side 203. The third wall portion 241 of the first wall side 201 may have substantially (±10%) the same height as the third wall portion 243 of the third wall side 203.

The inclination of the first wall portion 223 of the third wall side 203 may correspond or be equal to the inclination of the first wall portion 221 of the first wall side 201. The inclination of the second wall portion 233 of the third wall side 203 may correspond or be equal to the inclination of the second wall portion 231 of the first wall side 201. The inclination of the third wall portion 243 of the third wall side 203 may correspond or be equal to the inclination of the third wall portion 241 of the first wall side 201.

In general, the third wall side 203 may correspond to the first wall side 201 in such a way that the third wall side 203 is a reflection of the first wall side 201 with respect to the container axis 205. In general, the container axis 205 may be formed at the center of the bottom portion 210.

FIG. 8 shows the section D-D as indicated in FIG. 6, with the bottom portion 210, the second wall side 202 and the fourth wall side 204 being sectioned.

The second wall side 202 may include a first wall portion 222, a second wall portion 232, and a third wall portion 242.

The first wall portion 222 of the second wall side 202 may have the features of the first wall portion 221 of the first wall side 201. The second wall portion 232 of the second wall side 202 may have the features of the second wall portion 231 of the first wall side 201. The third wall portion 242 of the second wall side 202 may have the features of the third wall portion 241 of the first wall side 201.

The fourth wall side 204 may comprise a first wall portion 224, a second wall portion 234, and a third wall portion 244.

The first wall portion 224 of the fourth wall side 204 may have the features of the first wall portion 221 of the first wall side 201 The second wall portion 234 of the fourth wall side 204 may have the features of the second wall portion 231 of the first wall side 201. The third wall portion 244 of the fourth wall side 204 may have the features of the third wall portion 241 of the first wall side 201.

In general, the first wall side 201 of the container 200 may have the features of the first wall side 101 of the container 100. The second wall side 202 of the container 200 may have the features of the second wall side 102 of the container 100. The third wall side 203 of the container 200 may have the features of the third wall side 103 of the container 100. The fourth wall side 204 of the container 200 may have the features of the fourth wall side 104 of the container 100. Similarly, the wall sides 101, 102, 103, 104 of the container 100 may have the features of the wall sides 201, 202, 203, 204 of the container 200, respectively.

In general, an angle or inclination that is greater than another angle or inclination may be at least 1°, 2°, 3°, or 5° greater. In general, an angle or slope that is less than another angle or slope may be at least 1°, 2°, 3°, or 5° less.

FIG. 9 shows a system 400 for measuring a sample 320. The sample 320 may be placed in a container 100. A container 100 is indicated as an example in FIG. 9; a container 200 may also be used.

The sample 320 may have a height (in the direction of the container axis) of at least 2 mm, preferably at least 5 mm, more preferably at least 10 mm, more preferably at least 20 mm.

A liquid 330 may be introduced into the container 100. The liquid 330 may cover the sample 320 completely. The surface of the liquid 330 may be curved in an area of contact with a wall or walls of the container 100. A meniscus 331, 332 may form in the contact area. The liquid 330 may be placed in the container 100 such that the surface of the liquid 330 in parallel to a cross-sectional surface of the first wall portion or portions of the container 100 is substantially planar)(±5°. The cross-sectional surface of the first wall portion or the first wall portions of the container may be projected onto the surface of the liquid 330. Thus, an area at the surface of the liquid 330 may be considered that has the same dimensions as the cross-sectional surface of the first wall portion.

The system may include a radiation source 300. The radiation source 300 may be a laser. The sample 320 may be illuminated by the radiation source 300. The sample 320 is preferably illuminated by the radiation source 300 through a first wall portion of the container 100. An emission of the sample 320 may be stimulated by the illumination. The sample 320 may be illuminated through one of the wall sides, through at least two of the wall sides, or through all wall sides of the container. Different sides of the wall may be illuminated one after the other. At most one or two sides of the wall may be illuminated at the same time.

The system 400 may include a radiation receiver 350. The radiation receiver 350 may be a camera. The radiation receiver 350 may record the emission of the sample 320. The radiation source 300, the sample 320 and the radiation receiver 350 preferably do not lie on a (straight) line. The radiation receiver 350 may be arranged in such a way that an emission of the sample 320 can be recorded or is recorded substantially (±10°) perpendicular to the direction of illumination of the sample 320 with the radiation source 300. The radiation receiver 350 may be arranged in such a way that an emission of the sample 320 can be received or is received substantially (±10°) perpendicular to the bottom portion of the container 100.

An objective may be arranged between the sample 320 and the radiation receiver 350.

The objective may be at least partially immersed in the liquid 330 during illumination or measurement of the sample 320. The lens may also be positioned above the liquid surface while the sample 320 is being illuminated.

An opening of the container 100 may be covered during illumination of the sample 320, for example by one or more glass plates.

For example, the radiation source 300 may illuminate the sample 320 through a first wall portion of the container 100. Since the first wall side has an inclination of 1.9°±0.7° or n times that and due to the refractive indices of the interfaces on the path of the radiation from the radiation source 300 to the sample 320, the radiation from the radiation source 330 may be corrected in degrees (integer) such that the radiation is incident on the sample 320 parallel to the bottom portion or parallel to a liquid surface when the sample 320 is covered by a liquid. At the same time, the container may be manufactured inexpensively using an injection molding process (draft angles).

If the first wall portion of the third wall side of the container 100 is more inclined than the first wall portion of the wall side through which the illumination is performed, disruptive reflections can be avoided. Especially in the case of a small sample that is completely penetrated by the illumination of the radiation source 330, radiation can be reflected in the direction of the sample 330 when the first wall portion of the third wall side is not more inclined than the first wall portion of the wall side through which the illumination is performed.

The rectangular (non-square) basic shape of the container 100 or the bottom portion 110 of the container 100 may correspond to the optimal thin part (Rayleigh length) of a light sheet (radiation source 300) along the illumination direction and the field size of an objective in the direction perpendicular to the illumination direction.

Hereinafter, numbered exemplary embodiments are disclosed:
1. A container (100, 200) for a sample (320), said container (100, 200) comprising: a bottom portion (110, 210), wherein a container axis (105, 205) is oriented perpendicular to the bottom portion (110, 210); a first wall portion (121, 221), said first wall portion (121, 221) adjoining the bottom portion (110, 210); and a second wall portion (131, 231), said second wall portion (131, 231) adjoining said first wall portion (110, 210), and wherein said second wall portion (131, 231) is inclined by at least 20° with respect to said container axis (105, 205).
2. The container (100, 200) according to example 1, wherein the first wall portion (121, 221) is inclined by at least 0.5°, preferably by at least 1.0°, particularly preferably by at least 1.5° with respect to said container axis (105, 205); and/or wherein the first wall portion (121, 221) is inclined by at most 25°, preferably at most 10°, particularly preferably at most 5° with respect to the container axis (105, 205).
3. The container (100, 200) according to one of examples 1 or 2, wherein said container (100, 200) comprises a third wall portion (141, 241), said third wall portion (141, 241) adjoining said second wall portion (131, 231), and wherein said third wall portion (141, 241) is inclined by at least 2° with respect to said container axis (105, 205).
4. The container (100, 200) according to one of the preceding examples, wherein said container (100, 200) has a polygonal, in particular rectangular or square, basic shape and wherein said container (100, 200) comprises a first wall side (101, 201), a second wall side (102, 202), a third wall side (103, 203), and a fourth wall side (104, 204).
5. The container (100) according to example 4, wherein said first wall side (101) comprises the first, second and third wall portions (121, 131, 141), wherein said third wall side (103) comprises first, second and third wall portions (123, 133, 143), and wherein said first wall portion (123) of said third wall side (103) has a greater inclination with respect to said container axis (105) than said first wall portion (121) of said first wall side (101), in particular wherein said first wall portion (123) of said third wall side (103) has an inclination with respect to said container axis (105) that is greater than that of said first wall portion (121) of said first wall side (101) by at least 2°, preferably at least 5°.
6. The container (100) according to one of the examples 4 or 5, wherein said first wall side (101) comprises the first, second and third wall portions (121, 131, 141), wherein said third wall side (103) comprises first, second and third wall portions (123, 133, 143), and wherein said second wall portion (133) of said third wall side (103) has a smaller inclination with respect to said container axis (105) than said second wall portion (131) of said first wall side (101), in particular wherein said second wall portion (133) of said third wall side (103) has an inclination with respect to said container axis (105) that is less than that of said second wall portion (131) of said first wall side (101) by at least 2°, preferably at least 5°.
7. The container (100) according to one of examples 4 to 6, wherein said first wall side (101) comprises the first, second and third wall portions (121, 131, 141), wherein said second and/or fourth wall side (102, 104) each comprises first, second and third wall portions (122, 124, 132, 134, 142, 144), and wherein said first wall portion (122, 124) of said second wall side (102) and/or said fourth wall side (104) has a larger inclination with respect to said container axis (105) than said first wall portion (121) of said first wall side (101), in particular wherein said first wall portion (122, 124) of said second wall side (102) and/or said fourth wall side (104) has an inclination with respect to said container axis (105) that is greater than that of said first wall portion (121) of said first wall side (101) by at least one 1°, preferably at least 2°.
8. The container (100) according to one of examples 4 to 7, wherein said first wall side (101) comprises the first, second and third wall portions (121, 131, 141), wherein said second and/or fourth wall side (102, 104) each comprises first, second and third wall portions (122, 124, 132, 134, 142, 144), and wherein said second wall portion (132, 134) of said second wall side (102) and/or said fourth wall side (104) has a smaller inclination with respect to the container axis (105) than the second wall portion (131) of the first wall side (101), in particular wherein the second wall portion (132, 134) of the second wall side (102) and/or the fourth wall side (104) has an inclination with respect to the container axis (105) that is less than that of the second wall portion (131) of the first wall side (101) by at least one 1°, preferably at least 2°.
9. The container (200) according to example 4, wherein each of the first, second, third and fourth wall sides (201, 202, 203, 204) comprises said first wall portion (221) and/or said second wall portion (231) and/or said third wall portion (241).
10. A container (100, 200) for a sample (320), said container (100, 200) comprising: a bottom portion (110, 210), a container axis (105, 205) being oriented perpendicular to said bottom portion (110, 210); and a first wall portion (121, 221), said first wall portion (121, 221) adjoining said bottom portion (110, 210), and wherein said first wall portion (121, 221) is inclined with respect to said container axis (105, 205) by 1.9°±0.7° or n times that, where n is an integer greater than 1.
11. The container (100, 200) according to example 10, wherein said first wall portion (121, 221) is inclined with respect to said container axis (105, 205) by 1.9°±0.5°, in particular by 1.9°±0.3″, or n times that, where n is an integer and greater than 1.
12. The container (100, 200) according to one of the preceding examples, wherein said container (100, 200) is an injection molded container (100, 200) and/or said container (100, 200) comprises a thermoplastic, consists of a thermoplastic, and/or wherein said container (100, 200) comprises or consists of a block copolymer, in particular a cyclic block copolymer.
13. The container (100, 200) according to one of the preceding examples, wherein at least a portion of said container (100, 200) consists of a material, wherein said material has one or more of the following properties:
   a density, determined according to ASTM D792, of at most 1.00 g cm⁻³, preferably at most 0.98 g cm⁻³, more preferably at most 0.96 g cm⁻³, particularly preferably between 0.92 g cm⁻³ and 0.96 g cm⁻³; and/or
   a water absorption, determined according to ASTM D570, of less than 5%, preferably less than 2.0%, more preferably less than 0.5%; and/or
   a melt flow rate, determined according to ASTM D1238 at 2.6 kg and 260° C., of at least 1.0 cm⁻³ 10 min⁻¹, preferably at least 7.0 cm⁻³ 10 min⁻¹, more preferably at least 100.0 cm⁻³ 10 min⁻¹, more preferably between 2.0 cm⁻³ 10 min⁻¹ and 250.0 cm⁻³ 10 min⁻¹; and/or
   a transmittance, determined according to ASTM D1003, at 3 mm, of at least 50%, preferably at least 70%, more preferably at least 85%, more preferably at least 90%;
   a haze, determined according to ASTM D1003, at 3 mm, of less than 5.0%, preferably less than 3.0%, more preferably less than 1.5%, more preferably less than 1.0%; and/or
   a reflective index of less than 5.0, preferably less than 3.0, more preferably less than 2.5; and/or
   a Vicat softening point, determined according to ASTM D1525, at 1 kg, 50° C. h⁻¹, of at least 70° C., preferably at least 85° C., more preferably at least 100° C., more preferably between 90° C. and 130° C.; and/or
   a heat deflection temperature, determined according to ASTM D648, at 0.455 MPa, 2° C. min⁻¹, of at least 50° C., preferably at least 60° C., more preferably at least 75° C., more preferably between 60° C. and 115° C.; and/or
   a flexural strength, determined according to ASTM D790, of at least 30 MPa, preferably at least 40 MPa, more preferably at least 50 MPa, more preferably between 40 MPa and 80 MPa; and/or
   a flexural modulus, determined according to ASTM D790, of at least 600 MPa, preferably at least 1000 MPa, more preferably at least 1400 MPa, more preferably between 1000 MPa and 3000 MPa; and/or
   a tensile strength, Y.P. or B.P., determined according to ASTM D638, of at least 15 MPa, preferably at least 25 MPa, more preferably at least 30 MPa, more preferably between 20 MPa and 50 MPa; and/or
   a strain, determined according to ASTM D638, of at least 5%, preferably at least 8%, more preferably at least 10%, more preferably between 8% and 40%; and/or
   an Izod impact strength, determined according to ASTM D256, of at least 1 kg-cm cm⁻¹, preferably at least 1.5 kg-cm cm⁻¹, more preferably at least 2.0 kg-cm cm⁻¹, more preferably between 1.5 kg-cm cm⁻¹ and 5.0 kg-cm cm⁻¹; and/or
   a chemical resistance to methyl salicylate, dibenzyl ether, a mixture of one part benzyl alcohol and two parts benzyl benzoate, a mixture of four parts benzyl benzoate and one part diphenyl ether, ethyl cinnamate, and/or 2,2′-thiodiethanol; and/or
   an autofluorescence between 400 nm and 460 nm, at an excitation at 350 nm and a sample thickness of 1 mm, with respect to the autofluorescence of polystyrene of at most 50%, preferably at most 40%, more preferably at most 30%, more preferably at most 25%.
14. A method for examining a sample (320), in particular a biological sample, comprising the steps of:
   placing the sample (320) in a container (100, 200) according to one of the preceding examples;
   illuminating the sample (320); and
   recording an emission of the sample.
15. Use of a container (100, 200) according to one of examples 1 to 13 for storing a sample (320) during an examination of the sample (320), preferably during a microscopic examination, more preferably during a light sheet microscopic examination, more preferably during a light sheet fluorescence microscopic examination.

What is claimed is:

1. A container for a sample, comprising:
a bottom portion, a container axis being oriented perpendicular to said bottom portion;
a first wall portion, said first wall portion adjoining the bottom portion; and
a second wall portion, said second wall portion adjoining said first wall portion, and wherein said second wall portion is inclined by at least 20° with respect to said container axis,
wherein said container has a polygonal basic shape, and
material of the container is transparent.

2. The container of claim 1, wherein said first wall portion is inclined by at least 0.5° with respect to said container axis; and wherein said first wall portion is inclined by at most 25° with respect to the container axis.

3. The container of claim 1, wherein said container comprises a third wall portion, said third wall portion adjoining said second wall portion, and wherein said third wall portion is inclined by at least 2° with respect to said container axis.

4. The container of claim 3, wherein said container comprises a first wall side, a second wall side, a third wall side, and a fourth wall side.

5. The container of claim 4, wherein said first wall side comprises the first, second and third wall portions, wherein said third wall side comprises first, second and third wall portions, and wherein said first wall portion of said third wall side has a greater inclination with respect to said container axis than said first wall portion of said first wall side, wherein said first wall portion of said third wall side has an inclination with respect to said container axis that is greater than that of said first wall portion of said first wall side by at least 2°.

6. The container of claim 4, wherein said first wall side comprises the first, second and third wall portions, wherein said third wall side comprises first, second and third wall portions, and wherein said second wall portion of said third wall side has a smaller inclination with respect to said container axis than said second wall portion of said first wall side, wherein said second wall portion of said third wall side has an inclination with respect to said container axis that is less than that of said second wall portion of said first wall side by at least 2°.

7. The container of claim 4, wherein said first wall side comprises the first, second and third wall portions, wherein said second and/or fourth wall side each comprises first, second and third wall portions, and wherein said first wall portion of said second wall side and said fourth wall side has a larger inclination with respect to said container axis than said first wall portion of said first wall side, wherein said first wall portion of each of the second wall side and said fourth wall side has an inclination with respect to said container axis that is greater than that of said first wall portion of said first wall side by at least one 1°.

8. The container of claim 4, wherein said first wall side comprises the first, second and third wall portions, wherein said second and fourth wall sides each comprises first, second and third wall portions, and wherein said second wall portion of said second wall side and/or said fourth wall side has a smaller inclination with respect to the container axis than the second wall portion of the first wall side, wherein the second wall portion of each of the second wall side and the fourth wall side has an inclination with respect to said container axis that is less than that of the second wall portion of the first wall side by at least one 1°.

9. The container of claim 4, wherein each of the first, second, third and fourth wall sides comprises said first wall portion and said second wall portion and said third wall portion.

10. A container for a sample, said container comprising:
a bottom portion, a container axis being oriented perpendicular to said bottom portion; and
a first wall portion, said first wall portion adjoining said bottom portion, and wherein said first wall portion is inclined with respect to said container axis by 1.9°±0.3° or n times that, where n is an integer greater than 1, wherein said container has a polygonal basic shape.

11. The container of claim 10, wherein said container is an injection molded container and said container comprises a thermoplastic, consists of a thermoplastic, and/or wherein said container comprises or consists of a cyclic block copolymer.

12. The container of claim 10, wherein at least a portion of said container consists of a material, wherein said material has one or more of the following properties:
a density, determined according to ASTM D792, of at most 1.00 g cm⁻³;
a water absorption, determined according to ASTM D570, of less than 5%;
a melt flow rate, determined according to ASTM D1238 at 2.6 kg and 260° C., of at least 1.0 cm⁻³ 10 min⁻¹;
a transmittance, determined according to ASTM D1003, at 3 mm, of at least 50%;
a haze, determined according to ASTM D1003, at 3 mm, of less than 5.0%;
a reflective index of less than 5.0;
a Vicat softening point, determined according to ASTM D1525, at 1 kg, 50° C. h⁻¹, of at least 70° C.;
a heat deflection temperature, determined according to ASTM D648, at 0.455 MPa, 2° C. min′, of at least 50° C.;
a flexural strength, determined according to ASTM D790, of at least 30 MPa;
a flexural modulus, determined according to ASTM D790, of at least 600 MPa;
a tensile strength, Y.P. or B.P., determined according to ASTM D638, of at least 15 MPa;
a strain, determined according to ASTM D638, of at least 5%;
an Izod impact strength, determined according to ASTM D256, of at least 1 kg-cm cm⁻¹;
a chemical resistance to methyl salicylate, dibenzyl ether, a mixture of one part benzyl alcohol and two parts benzyl benzoate, a mixture of four parts benzyl benzoate and one part diphenyl ether, ethyl cinnamate, and 2,2′-thiodiethanol; and
an autofluorescence between 400 nm and 460 nm, at an excitation at 350 nm and a sample thickness of 1 mm, with respect to the autofluorescence of polystyrene of at most 50%.

13. A method for examining a biological sample, comprising:
placing the sample in the container of claim 1;
illuminating the sample; and
recording an emission of the sample, wherein the emission of the sample is recorded substantially perpendicular to a direction of illumination.
